(19) United States
(12) Patent Application Publication
(10) Pub. No.: US 2002/0150901 A1
(43) Pub. Date: Oct. 17, 2002

(54) Novel nucleic acids, polypeptides, methods of making, and uses thereof

(76) Inventors: Andrew Murphy, Croton-on-Hudson, NY (US); Joel Martin, Putnam Valley, NY (US)

Correspondence Address: Linda O. Palladino, Regeneron Pharmaceuticals, Inc., 777 Old Saw Mill River Road, Tarrytown, NY, 10591, US

(21) Appl. No.: 09/866,230

(22) Filed: May 25, 2001

Related U.S. Application Data
(60) Provisional application No. 60/207,725, filed on May 26, 2000.

(51) Int. Cl. C12Q 1/68; C07K 1/00; C07K 14/00; C07K 17/00

(52) U.S. Cl. 435/6; 530/350

(57) ABSTRACT

HUMAN CysLT2 GPCR polypeptides and related nucleic acids are provided. Included are natural HUMAN CysLT2 GPCR homologs from several species and polypeptides comprising a HUMAN CysLT2 GPCR domain having specific activity. The polypeptides may be produced recombinantly from transformed host cells with the subject nucleic acids. Also provided are isolated hybridization probes and oligonucleotide primers capable of specifically hybridizing with the disclosed genes, specific binding agents and methods of making and using the subject compositions, including high throughput screens.

This application claims priority to U.S. Provisional Application No. 60/207,725, filed May 26, 2000. All publications and patent applications cited in this specification are hereby incorporated by reference as if each individual publication or patent application were specifically and individually indicated to be incorporated by reference.

### FIELD OF THE INVENTION

The field of this invention is nucleic acids and polypeptides which are G-protein-coupled receptor (GPCR) nucleic acids and polypeptides, and in particular, HUMAN CysLT2 GPCR nucleic acids and polypeptides, as well as methods of making said polypeptides and methods of using said nucleic acids and polypeptides.

### BACKGROUND OF THE INVENTION

G-protein coupled receptors (GPCRs) are a class of integral membrane proteins which contain seven hydrophobic transmembrane domains that span the cell membrane and form a cluster of anti-parallel alpha helices. These seven transmembrane domains provide many of the structural and functional features of the GPCR. The cluster of alpha helices forms a pocket into which a low-molecular-weight ligand can bind. In instances when the pocket must accommodate a larger ligand (e.g. a peptide or protein), either the extracellular N-terminal portion of the GPCR or one or more of the three extracellular loops participate in binding the ligand. In some cases (e.g. metabotropic glutamate receptors, Ca²⁺sensing receptors and glycoprotein hormone receptors) a large extracellular amino-terminus of the receptor binds a ligand, and then this complex presumably binds to the extracellular loops to activate the receptor. In yet another mode of activation, some receptors (the protease activated receptors, e.g. PAR1, PAR2, PAR3 and PAR4) are activated by cleavage of their extracellular amino-terminus. In this case the new amino-terminus generated by cleavage serves as a tethered ligand that binds to and activates the receptor. It is the binding of a ligand that activates the GPCR by triggering conformational changes in intracellular portions of the GPCR. Once activated, a GPCR interacts with an intracellular heterotrimeric G-protein causing it to release GDP and bind GTP as well as dissociating the α subunit from the βγ subunit heterodimer. The activated α-GTP complex and free βγ moieties mediate additional intracellular signaling, often including the activation of effector enzymes (e.g. adenylyl cyclase (Sunahara R K, et al., Annu Rev Pharmacol Toxicol. 1996,36:461-80); phospholipase-Cβ (Morris A J; Scarlata S, Biochem Pharmacol Aug. 15, 1997;54(4):429-35); and P13-kinase (Zhong Li, Huiping Jiang, Wei Xie, Zuchuan Zhang, Alan V. Smrcka, and Dianqing Wu, Science 2000 287: 1046-1049; Guy Servant, Orion D. Weiner, Paul Herzmark, Tamas Balla, John W. Sedat, and Henry R. Bourne, Science 2000 287: 1037-1040)) that result in the production of intracellular second messengers, for example, cyclic AMP (cAMP), inositol (1,4,5) triphosphate, or phosphatidylinositol (3,4,5) triphosphate (Baldwin, J. M. (1994) Curr. Opin. Cell Biol. 6:180-190). Free βγ also has other effects, for instance the activation of K+channels and inhibition of Ca²⁺ channels (Clapham D E and Neer E J, Annu Rev Pharmacol Toxicol 1997;37:167-203).

As stated supra, the N-terminus of GPCRs is located extracellularly. Its length can vary and it may or may not be glycosylated. The C-terminus is located intracellularly and is often phosphorylated upon activation. Alternating extracellular and intracellular loops connect the seven transmembrane domains (See Baldwin J M; Schertler G F; Unger V M, J Mol Biol Sep. 12, 1997;272(1):144-64 for a general review of GPCR structure).

GPCRs respond to many different types of ligands including, but not limited to, lipid analogs, amino acids and their derivatives, polypeptides, hormones and chemokines. In addition, GPCRs are able to respond to specialized types of stimuli such as light, taste, and odor. For example, GPCRs function in physiological processes including vision (the rhodopsins), smell (the olfactory receptors), neurotransmission (serotonin, metabotropic glutamate, GABA-B, muscarinic acetylcholine, dopamine, and adrenergic receptors), and hormonal responses (luteinizing hormone and thyroid-stimulating hormone receptors).

In accordance with the present invention, Applicants disclose herewith a novel, newly discovered GPCR that exhibits sequence homology to the previously described cysteinyl leukotriene receptor termed CysLT1 (Sarau H M, Ames R S, Chambers J, Ellis C, Elshourbagy N, Foley J J, Schmidt D B, Muccitelli R M, Jenkins O, Murdock P R, Herrity N C, Halsey W, Sathe G, Muir A I, Nuthulaganti P, Dytko G M, Buckley P T, Wilson S, Bergsma D J, Hay D W, Mol Pharmacol. 1999 September;56(3):657-63; Lynch K R, O'Neill G P, Liu Q, Im D S, Sawyer N, Metters K M, Coulombe N, Abramovitz M, Figueroa D J, Zeng Z, Connolly B M, Bai C, Austin C P, Chateauneuf A, Stocco R, Greig G M, Kargman S, Hooks S B, Hosfield E, Williams D L Jr, Ford-Hutchinson A W, Caskey C T, Evans J F, Nature. Jun. 24, 1999;399(6738):789-93.; GenBank accession number: NP_{—}006630). The identification, sequencing and characterization of this new molecule, termed HUMAN CysLT2 GPCR, and the nucleic acids encoding it provide new compositions which are useful in the diagnosis, prevention and treatment of inflammatory, immunological, vascular or other disorders and in the development of assays to screen for molecules that modulate HUMAN CysLT2 GPCR activity. In addition, the discovery that the cysteinyl leukotrienes, LTC4 and LTD4 are agonists of this receptor will be useful in development of screens and the synthesis of additional compounds that modulate the behavior of this receptor.

The previously cloned CysLT1 receptor is expressed in eosinophils as well as bronchiolar smooth muscle and is the target of the recently approved asthma medications montelukast (Singulair/MK-0476) (Jones T R, Labelle M, Belley M, Champion E, Charette L, Evans J, Ford-Hutchinson A W, Gauthier J Y, Lord A, Masson P, et al., Can J Physiol Pharmacol. 1995 February;73(2):191-201.), zafirlukast (Accolate/ICI 204,219)(Krell R D; Dehaas C J; Lengel D J; Kusner E J; Williams J C; Buckner C K, Ann N Y Acad Sci Nov. 15,1994;744:289-98) and pranlukast (Onon/ONO-1078)(Grossman J, Faiferman I, Dubb J W, Tompson D J, Busse W, Bronsky E, Montanaro A, Southern L, Tinkelman D, J Asthma. 1997;34(4):321-8.). CysLT2 receptor was found on human pulmonary veins (Labat C, et al., J Pharmacol Exp Ther 1992 263:800) and several animal smooth muscle preparations (Back M; Jonsson E W; Dahlen S E, Eur J Pharmacol Dec. 12, 1996;317(1):107-13; Jonsson E W, Pulm Pharmacol Ther. 1997;10(1):29-36). Applicants have also observed that the HUMAN CysLT2 GPCR that is the subject of this invention exhibits several of functional characteristics including greater activity with LTC4 than with LTD4 and resistance to inhibition by MK571 and Singulair of CysLT2. Applicants have identified, cloned and sequenced a novel GPCR and, because the Applicants' discovery represents only the second cysteinyl leukotriene receptor actually cloned and defined as such, the Applicants have termed this receptor HUMAN CysLT2 GPCR. In addition, a non-CysLT1 cysteinyl leukotriene has been reported in human endothelial cells (Pedersen K E, Bochner B S, Undem B J, J Pharmacol Exp Ther. 1997 May;281(2):655-62). This molecule presumably mediates the vascular-leakage effects of LTC4 (Hua X Y; Dahlen S E; Lundberg J M; Hammarstrom S; Hedqvist P, Naunyn Schmiedebergs Arch Pharmacol 1985 August;330(2):136-41). HUMAN CysLT2 GPCR may be the receptor mediating these effects, making it likely that HUMAN CysLT2 GPCR inhibitors would be active agents in inhibiting edemous conditions.

### SUMMARY OF THE INVENTION

The subject invention provides for isolated HUMAN CysLT2 GPCR polypeptides and polypeptides comprising a fragment or derivative thereof.

The subject invention also provides for isolated HUMAN CysLT2 GPCR polypeptides, comprising the amino acid sequence as set forth in FIGS. 1A-1C.

The subject invention further provides for the isolated HUMAN CysLT2 GPCR polypeptides encoded by the nucleic acid molecule as set forth in FIGS. 1A-1C.

Another embodiment of the invention is a vector which comprises nucleic acid molecules as set forth in FIGS. 1A-1C.

A further embodiment is a vector wherein the nucleic acid molecule is operatively linked to an expression control sequence capable of directing its expression in a host cell.

In yet another embodiment the vector is a plasmid.

An additional embodiment of the invention is a chimeric protein which comprises the extracellular portions of the CysLT2 GPCR protein fused to an immunoglobulin, an immunoglobulin constant region or a fragment thereof. Such extracellular portions may comprise the amino terminus of CysLT2, the amino acids located between the second and third transmembrane domains, the amino acids located between the fourth and fifth transmembrane domains, the amino acids located between the fifth and sixth transmembrane domains, or any combination thereof. The extracellular amino terminus comprises amino acid residues 1-41 of FIGS. 1A-1C, the extracellular region between the second and third transmembrane domains comprises amino acid residues 94-115 of FIGS. 1A-1C, the extracellular region between the fourth and fifth transmembrane domains comprises amino acid residues 175-203 of FIGS. 1A-1C, and the extracellular region between the sixth and seventh transmembrane domains comprises amino acid residues 268-289 of FIGS. 1A-1C.

Another embodiment of the invention is a host-vector system for the production of HUMAN CysLT2 GPCR which comprises a vector in a host cell wherein the host cell is a bacterial, yeast, insect, amphibian or mammalian cell.

The invention further contemplates a method of producing HUMAN CysLT2 GPCR which comprises growing cells of a host-vector system under conditions permitting production of the HUMAN CysLT2 GPCR, and recovering the HUMAN CysLT2 GPCR so produced.

Still another embodiment of the invention provides for an antibody which specifically binds the HUMAN CysLT2 GPCR polypeptide. The antibody may be a polyclonal antibody or a monoclonal antibody, including a wholly human monoclonal antibody.

The invention provides for a composition comprising HUMAN CysLT2 GPCR polypeptide and a carrier as well as a composition comprising an antibody and a carrier wherein the compositions are for use in a method of treatment of the human or animal body, or in a method of diagnosis.

Another embodiment of the invention provides a method of identifying a HUMAN CysLT2 GPCR binding target comprising (a) contacting HUMAN CysLT2 GPCR polypeptide with a test sample suspected of containing a HUMAN CysLT2 GPCR binding target; (b) contacting HUMAN CysLT2 GPCR polypeptide with a control sample that does not contain a HUMAN CysLT2 GPCR binding target (c) comparing the amount of binding in (a) to the amount of binding in (b) wherein a greater amount of binding in (a) is indicative of the presence of a HUMAN CysLT2 GPCR binding target in the test sample.

Another embodiment of the invention provides a method of identifying modulators of HUMAN CysLT2 GPCR function using a ligand displacement assay. In such an assay potential modulators are identified by incubating a test sample with the HUMAN CysLT2 GPCR protein and a known labeled binding partner (e.g. LTC4 or LTD4 ). The amount of the known labeled binding partner which binds to the HUMAN CysLT2 GPCR protein in the mixture is determined and compared to the amount which binds in a parallel reaction lacking the test sample. A reduction in the amount of known labeled binding partner in the presence of the test sample compared to the parallel reaction indicates the presence of a HUMAN CysLT2 GPCR modulator in the test sample. Modulators in this assay can be either agonists or antagonists.

Another embodiment of the invention provides a method of identifying modulators of HUMAN CysLT2 GPCR function using a biological readout in HUMAN CysLT2 GPCR expressing cells or cell fragments. Agonists are identified by incubating cells or cell fragments engineered to express the HUMAN CysLT2 GPCR protein with test samples and measuring a biological response in these cells and in parallel cells or cell fragments not expressing the HUMAN CysLT2 GPCR protein. An increased biological response in the cells or cell fragments expressing the HUMAN CysLT2 GPCR protein compared to the parallel cells or cell fragments indicates the presence of an agonist in the test sample. Likewise, antagonists are identified by incubating cells or cell fragments engineered to express the HUMAN CysLT2 GPCR protein with test samples in the presence of a known HUMAN CysLT2 GPCR agonist (e.g. LTC4 or LTD4). The amount of biological response is measured and compared to a parallel reaction lacking the test sample. A reduction of the biological response in the presence of the test sample compared to the parallel reaction indicates the presence of an antagonist.

The subject invention provides for unique polypeptides called HUMAN CysLT2 GPCR encoded by nucleic acids as set forth in FIGS. 1A-1C which were identified by screening virtual proteins derived from the NCBI human genomic sequence database with sequences obtained from known and predicted family members.

The invention comprises nucleic acids which are complementary to the HUMAN CysLT2 GPCR sequences as set forth in FIGS. 1A-1C.

The invention also comprises the use of HUMAN CysLT2 GPCR sequences to identify and obtain a full length HUMAN CysLT2 GPCR cDNA.

The invention further comprises the use of oligomers from the HUMAN CysLT2 GPCR sequence in a HUMAN CysLT2 GPCR kit which can be used to identify a disorder or disease with altered HUMAN CysLT2 GPCR expression and provide a method for monitoring progress of a patient during drug therapy.

In addition, the invention comprises the use of HUMAN CysLT2 GPCR-specific antibodies in assays to identify a disorder or disease with altered HUMAN CysLT2 GPCR expression and provides a method to monitor the progress of a patient during drug therapy.

Another embodiment of the invention is a competitive binding assay useful for identifying an agent which specifically binds to HUMAN CysLT2 GPCR comprising a) obtaining cells expressing on their cell surface HUMAN CysLT2 GPCR, b) contacting the cells with a first agent known to bind to HUMAN CysLT2 GPCR, c) detecting the amount of binding of the first agent in (b) to HUMAN CysLT2 GPCR, d) contacting (b) with a second agent whose ability to specifically bind to HUMAN CysLT2 GPCR is unknown, e) detecting the amount of binding of the first agent in (d) to HUMAN CysLT2 GPCR, and f) comparing the amount of binding of the first agent detected in (c) with the amount of binding of the first agent detected in (e) wherein a decrease in the amount of binding of the first agent is indicative of the second agent's ability to specifically bind to HUMAN CysLT2 GPCR.

Another embodiment of the invention is a competitive binding assay useful for identifying an agent which specifically binds to HUMAN CysLT2 GPCR comprising a) preparing a sample comprising HUMAN CysLT2 GPCR, b) contacting the sample with a first agent known to bind to HUMAN CysLT2 GPCR. c) detecting the amount of binding of the first agent in (b) to HUMAN CysLT2 GPCR, d) contacting (b) with a second agent whose ability to specifically bind to HUMAN CysLT2 GPCR is unknown, e) detecting the amount of binding of the first agent in (d) to HUMAN CysLT2 GPCR, and f) comparing the amount of binding of the first agent detected in (c) with the amount of binding of the first agent detected in (e) wherein a decrease in the amount of binding of the first agent is indicative of the second agent's ability to specifically bind to HUMAN CysLT2 GPCR.

Another embodiment of the invention is a competitive binding assay useful for identifying an agent which specifically binds to HUMAN CysLT2 GPCR comprising a) obtaining cells expressing on their cell surface HUMAN CysLT2 GPCR, b) contacting a test sample of the cells of (a) with a first agent whose ability to specifically bind to HUMAN CysLT2 GPCR is unknown, c) contacting the test sample of the cells of (b) with a second agent known to bind to HUMAN CysLT2 GPCR, d) contacting a control sample of the cells of (a) with the second agent known to bind to HUMAN CysLT2 GPCR, e) detecting the amount of binding of the second agent in (c) to HUMAN CysLT2 GPCR, f) detecting the amount of binding of the second agent in (d) to HUMAN CysLT2 GPCR, and g) comparing the amount of binding of the second agent detected in (e) with the amount of binding of the second agent detected in (f) wherein a lesser amount of binding of the second agent in (e) is indicative of the first agent's ability to specifically bind to HUMAN CysLT2 GPCR.

Another embodiment of the invention is a competitive binding assay useful for identifying an agent which specifically binds to HUMAN CysLT2 GPCR comprising a) preparing a sample comprising HUMAN CysLT2 GPCR, b) contacting a test sample of the sample of (a) with a first agent whose ability to specifically bind to HUMAN CysLT2 GPCR is unknown, c) contacting the test sample of (b) with a second agent known to bind to HUMAN CysLT2 GPCR, d) contacting a control sample of the sample of (a) with the second agent known to bind to HUMAN CysLT2 GPCR, e) detecting the amount of binding of the second agent in (c) to HUMAN CysLT2 GPCR, f) detecting the amount of binding of the second agent in (d) to HUMAN CysLT2 GPCR, and g) comparing the amount of binding of the second agent detected in (e) with the amount of binding of the second agent detected in (f) wherein a lesser amount of binding of the second agent in (e) is indicative of the first agent's ability to specifically bind to HUMAN CysLT2 GPCR.

Another embodiment of the invention is a competitive binding assay useful for identifying an agent which specifically binds to HUMAN CysLT2 GPCR comprising a) obtaining cells expressing on their cell surface HUMAN CysLT2 GPCR, b) contacting a test sample of the cells of (a) with a first agent known to bind to HUMAN CysLT2 GPCR and with a second agent whose ability to bind to HUMAN CysLT2 GPCR is unknown, c) contacting a control sample of the cells of (a) with the first agent known to bind to HUMAN CysLT2 GPCR, d) detecting the amount of binding of the first agent in (b) to HUMAN CysLT2 GPCR, e) detecting the amount of binding of the first agent in (c) to HUMAN CysLT2 GPCR, and f) comparing the amount of binding of the first agent detected in (d) with the amount of binding of the first agent detected in (e) wherein a decrease in the amount of binding of the first agent in (d) is indicative of the second agent's ability to specifically bind to HUMAN CysLT2 GPCR.

Another embodiment of the invention is a competitive binding assay useful for identifying an agent which specifically binds to HUMAN CysLT2 GPCR comprising a) preparing a sample comprising HUMAN CysLT2 GPCR, b) contacting a test sample of the sample of (a) with a first agent known to bind to HUMAN CysLT2 GPCR and with a second agent whose ability to bind to HUMAN CysLT2 GPCR is unknown, c) contacting a control sample of the sample of (a) with the first agent known to bind to HUMAN CysLT2 GPCR, d) detecting the amount of binding of the first agent in (b) to HUMAN CysLT2 GPCR, e) detecting the amount of binding of the first agent in (c) to HUMAN CysLT2 GPCR, and f) comparing the amount of binding of the first agent detected in (d) with the amount of binding of the first agent detected in (e) wherein a decrease in the amount of binding of the first agent in (d) is indicative of the second agent's ability to specifically bind to HUMAN CysLT2 GPCR.

In particular embodiments of the invention the detection of specific binding of the agent to HUMAN CysLT2 GPCR is accomplished by any one of the methods selected from the group consisting of radioactive detection, fluorescence detection, chromogenic detection, mass spectroscopy, and plasmon resonance.

In another preferred embodiment of the invention, the first agent is LTC4 or LTD4 or a derivative thereof and in yet another embodiment the second agent is LTC4 or LTD4 or a derivative thereof.

In another particular embodiment of the invention the detection of specific binding of the agent to HUMAN CysLT2 GPCR is accomplished by detecting a biological response wherein the biological response is selected from the group consisting measuring Ca²⁺ ion flux, cAMP, IP₃, PIP₃and transcription of reporter genes. Suitable reporter genes include endogenous genes as well as exogenous genes that are introduced into a cell by any of the standard methods familiar to the skilled artisan, such as transfection, electroporation, lipofection and viral infection.

In a further embodiment of the invention the cell expressing HUMAN CysLT2 GPCR is a mammalian cell and in a particular embodiment the mammalian cell is a COS-7 cell, a 293 human embryonic kidney cell, a NIH 3T3 cell, or Chinese hamster ovary (CHO) cell.

### BRIEF DESCRIPTION OF THE FIGURES

FIGS. 1A-1C. The nucleic acid sequences (sense and antisense) and deduced amino acid sequence of HUMAN CysLT2 GPCR.

FIG. 2. A sequence comparison of CysLT1 and HUMAN CysLT2 GPCR. The high degree of homology, especially within the transmembrane domains, indicates that HUMAN CysLT2 GPCR is a putative GPCR family member. Transmembrane segments are underlined (from alignment to Baldwin model-JMB (97) 272:144-164). Potential N-linked glycosylation sites are double underlined. Cysteine residues on the extracellular face are enboldened and italicized

FIGS. 3A-3B. Expression pattern of HUMAN CysLT2 GPCR using TaqMan analysis. The CysLT2 gene is broadly expressed, but most abundant expression is seen in adrenal gland, placenta and spleen. It is also weakly expressed in several hematopoietic cell lines.

FIG. 4. Activation of HUMAN CysLT2 GPCR by LTC4 and LTD4. COS-7 cells expressing HUMAN CysLT2 GPCR, human CysLT1 or no leukotriene receptor and aequorin were assayed for aequorin activation in response to 200 nM of LTC4 or LTD4, either alone, or in the presence of 200 nM of the CysLT1 antagonists MK571 or Singulair (Montelukast). A signal was detected from both agonists on both CysLT receptors but not on the “dummy” transfected control cells. The CysLT1 antagonists failed to block HUMAN CysLT2 GPCR activation while they effectively blocked CysLT1.

### DEFINITIONS

An “oligonucleotide” or “oligomer” is a stretch of nucleotide residues which has a sufficient number of bases to be used in a polymerase chain reaction (PCR). These short sequences are based on (or designed from) genomic or cDNA sequences and are used to amplify, confirm, or reveal the presence of an identical, similar or complementary DNA or RNA in a particular cell or tissue or test sample. Oligonucleotides or oligomers comprise portions of a DNA sequence having at least about 10 nucleotides and as many as about 50 nucleotides, preferably about 15 to 30 nucleotides. They are chemically synthesized and may be used as probes.

“Probes” are nucleic acid sequences of variable length, preferably between at least about 10 and as many as about 6,000 nucleotides, depending on use. They are used in the detection of identical, similar, or complementary nucleic acid sequences in a particular cell or tissue or test sample. Longer length probes are usually obtained from a natural or recombinant source, are highly specific and much slower to hybridize than oligomers. They may be single- or double-stranded and carefully designed to have specificity in PCR, hybridization membrane-based, or ELISA-like technologies.

“Reporter” molecules are chemical moieties used for labeling a nucleic or amino acid sequence. They include, but are not limited to, radionuclides, enzymes, fluorescent, chemi-luminescent, or chromogenic agents. Reporter molecules associate with, establish the presence of, and may allow quantification of a particular nucleic or amino acid sequence.

“Reporter genes” include endogenous genes as well as exogenous genes that are introduced into a cell by any of the standard methods familiar to the skilled artisan, such as transfection, electroporation, lipofection, and viral infection.

A “portion” or “fragment” of a nucleic acid comprises all or any part of the nucleic acid sequence having fewer nucleotides than about 6 kb, preferably fewer than about 1 kb. Such portions or fragments may be used as probes may be labeled with reporter molecules using nick translation, Klenow fill-in reaction, PCR or other methods well known in the art. After pretesting to optimize reaction conditions and to eliminate false positives, nucleic acid probes may be used in Southern, northern or in situ hybridizations to determine whether DNA or RNA encoding the protein is present in a biological sample, cell type, tissue, organ or organism. The portions or fragments may also be used to construct fusion molecules. These fusion molecules may be made by fusing a nucleic acid encoding a first polypeptide with a nucleic acid encoding a second polypeptide such that the final fused nucleic acid encodes a chimeric polypeptide.

“Recombinant nucleotide variants” are nucleic acids which encode a protein. They may be synthesized by making use of the “redundancy” in the genetic code. Various codon substitutions, such as the silent changes which produce specific restriction sites or codon usage-specific mutations, may be introduced to optimize cloning into a plasmid or viral vector or expression in a particular prokaryotic or eukaryotic host system, respectively.

“Control elements” or “regulatory sequences” or “expression control sequences” are those nontranslated regions of the gene or DNA such as enhancers, promoters, introns and 3′ untranslated regions which interact with cellular proteins to carry out replication, transcription, and translation. They may occur as boundary sequences or even split the gene. They function at the molecular level and along with regulatory genes are very important in development, growth, differentiation and aging processes.

“Chimeric” or “fusion” molecules are nucleic acids or polypeptides which are created by fusing or combining one or more of nucleic acid sequences of this invention (or their parts) with additional nucleic acid sequence(s). Such fused or combined sequences may be introduced into an appropriate vector and expressed to give rise to a chimeric polypeptide which may be expected to be different from the native molecule in one or more of the following characteristics: cellular location, distribution, ligand-binding affinities, interchain affinities, degradation/turnover rate, signaling, etc.

“Active” is that state in which a polypeptide is capable of being useful or of carrying out some role or function. In the subject application, it specifically refers to those forms, fragments, or domains of a polypeptide sequence which display the biologic and/or immunogenic activity characteristic of the naturally occurring HUMAN CysLT2 GPCR.

“Naturally occurring HUMAN CysLT2 GPCR” refers to a polypeptide produced by cells which have not been genetically engineered or which have been genetically engineered to produce the same sequence as that which is naturally produced. Specifically contemplated by the invention are various polypeptides which arise from post-translational modifications. Such modifications of the polypeptide include, but are not limited to, acetylation, carboxylation, glycosylation, phosphorylation, lipidation and acylation.

“Derivative” refers to those polypeptides which have been chemically modified by such techniques as ubiquitination, labeling (see above), pegylation (derivatization with polyethylene glycol), and chemical insertion or substitution of amino acids such as ornithine which do not normally occur in human proteins.

“Recombinant polypeptide variant” refers to any polypeptide which differs from naturally occurring HUMAN CysLT2 GPCR by amino acid insertions, deletions and/or substitutions, created using recombinant DNA techniques. Guidance in determining which amino acid residues may be replaced, added or deleted without abolishing characteristics of interest may be found by comparing the sequence of HUMAN CysLT2 GPCR with that of related polypeptides and minimizing the number of amino acid sequence changes made in highly conserved regions.

Amino acid “substitutions” are defined as one-for-one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine. Non-conservative substitutions involve replacement with an amino acid that has significantly different structural and/or chemical properties than the amino acid residue it is replacing.

Amino acid “insertions” or “deletions” are changes to or within an amino acid sequence. They typically fall in the range of about 1 to 5 amino acids. The variation allowed in a particular amino acid sequence may be experimentally determined by producing the peptide synthetically or by systematically making insertions, deletions, or substitutions of nucleotides in the HUMAN CysLT2 GPCR sequence using recombinant DNA techniques.

A “signal or leader sequence” or “signal peptide” is a short amino acid sequence which or can be used, when desired, to direct the polypeptide through a membrane of a cell. Such a sequence may be naturally present on the polypeptides of the present invention or provided from heterologous sources by recombinant DNA techniques.

An “oligopeptide” is a short stretch of amino acid residues and may be expressed from an oligonucleotide. It may be functionally equivalent to and either the same length as or considerably shorter than a “fragment”, “portion ”, or “segment” of a polypeptide. Such sequences comprise a stretch of amino acid residues of at least about 5 amino acids and often about 17 or more amino acids, typically at least about 9 to 13 amino acids, and of sufficient length to display biologic and/or immunogenic activity.

An “inhibitor” is a substance which retards or prevents a chemical or physiological reaction or response. Common inhibitors include but are not limited to antisense molecules, antibodies, antagonists and their derivatives.

An “agonist” is a substance that causes activation of a receptor as measured by any of a number or biological or biochemical readouts.

An “antagonist” is a substance which prevents activation or retards the activation of a receptor by an agonist.

A “standard” is a quantitative or qualitative measurement for comparison. Preferably, it is based on a statistically appropriate number of samples and is created to use as a basis of comparison when performing diagnostic assays, running clinical trials, or following patient treatment profiles. The samples of a particular standard may be normal or similarly abnormal.

“Animal” as used herein may be defined to include human, domestic (cats, dogs, etc.), agricultural (cows, horses, sheep, goats, chicken, fish, etc.) or test species (amphibian, frogs, mice, rats, rabbits, simians, etc.).

“Disorders or diseases” in which altered HUMAN CysLT2 GPCR activity have been implicated specifically include, but are not limited to, reproductive diseases, diseases related to cellular metabolism, growth, development, blood and bone homeostasis.

Since the list of technical and scientific terms cannot be all encompassing, any undefined terms shall be construed to have the same meaning as is commonly understood by one of skill in the art to which this invention belongs. Furthermore, the singular forms “a”, “an” and “the” include plural referents unless the context clearly dictates otherwise. For example, reference to a “restriction enzyme” or a “high fidelity enzyme” may include mixtures of such enzymes and any other enzymes fitting the stated criteria, or reference to the method includes reference to one or more methods for obtaining cDNA sequences which will be known to those skilled in the art or will become known to them upon reading this specification.

Before the present sequences, variants, formulations and methods for making and using the invention are described, it is to be understood that the invention is not to be limited only to the particular sequences, variants, formulations or methods described. The sequences, variants, formulations and methodologies may vary, and the terminology used herein is for the purpose of describing particular embodiments. The terminology and definitions are not intended to be limiting since the scope of protection will ultimately depend upon the claims.

### DETAILED DESCRIPTION OF THE INVENTION

The subject Application provides for the identification of a novel member of the GPCR family of receptors called HUMAN CysLT2 GPCR. The invention further provides efficient methods of identifying agents, compounds or lead compounds for agents active at the level of HUMAN CysLT2 GPCR modulatable cellular function. Generally, these screening methods involve assaying for compounds which either activate on their own or modulate HUMAN CysLT2 GPCR interaction with a natural (e.g. LTC4 or LTD4) or synthetic HUMAN CysLT2 GPCR binding target. A wide variety of assays for binding agents are provided including, but not limited to, protein-protein binding assays, immunoassays, and cell based assays. Preferred methods are amenable to automated, cost-effective high throughput screening of chemical libraries for lead compounds. An automated, cost effective high throughput screen may be performed in a number of ways. For example, a competitive binding assay in which the ability of a test compound to displace the binding of LTC4, LTD4 or their derivatives from the HUMAN CysLT2 GPCR receptor expressed on membranes using any of a number of transfected expression systems (COS-7, HEK293, CHO or other tissue culture cells, baculovirus, yeast including Saccharomyces cerevisiae, Pichia pastoris, or others) is measured using LTC4, LTD4 or one of their derivatives labeled with, for example, a radioactive or fluorescent tag. The binding of HUMAN CysLT2 GPCR to labeled LTC4, LTD4 or their derivatives can be measured by physical separation of HUMAN CysLT2 GPCR expressing membranes from the soluble, labeled LTC4, LTD4 or derivatives. Such separation may be accomplished using centrifugation or filtration. In a preferred embodiment the binding can be detected by one of several “homogeneous” methods that do not rely of physical separation. Such methods might include scintillation proximity assay (SPA) (Hart H E, Greenwald E B, Mol Immunol. 1979 April;16 (4):265-7), fluorescence resonance energy transfer (FRET) (e.g. EP 103,558, U.S. Pat. No. 4,587,223) or fluorescence polarization.

A high throughput screen may also be established by detecting the activation (test compound vs. no test compound) or inhibition (test compound vs. no test compound, either in the presence of an agonist, for example LTC4 or LTD4, or a HUMAN CysLT2 GPCR receptor activated by mutation or overexpression) of a biological response in HUMAN CysLT2 GPCR transfected cells. Such cells could include mammalian cell lines (e.g. COS-7, HEK293, CHO 3T3), insect cells (e.g. Schneider, sf9, hi5), frog melanophore cells, Saccharomyces cerevisiae, or other suitable cells. Biological readouts might include calcium flux measured by changes in fluorescence of a calcium sensing fluorophore (e.g. FURA2 or a chameleon (Miyawaki A, Llopis J, Heim R, McCaffery J M, Adams J A, Ikura M, Tsien R Y, Nature. Aug. 28, 1997;388(6645):882-7)) on a FLIPR (Fluorescence Imaging Plate Reader) or by light emission of a protein which emits light in a calcium-dependent manner (e.g. aequorin, see infra for a description of this assay).

Other biological readouts include direct measurement of second messengers. For instance, increases in cAMP levels or decreases in forskolin stimulated cAMP levels can be measured using standard cAMP RIA, standard competition ELISA or SPA. Increases in the second messengers IP3 (PLC activation) and PIP3 (PI3K activation) can also serve as a measure of receptor activation using similar systems. Additional biological readouts include transcriptional activation readouts by either direct measurement of mRNA levels or through the use of enzymatic reporter genes (for example, luciferase, beta-galactosidase or beta-lactamase). Other biological readouts include cell proliferation and changes in pigment distribution (see, for example, McClintock T S, Graminski G F, Potenza M N, Jayawickreme C K, Roby-Shemkovitz A and Lerner MR (1993), Anal Biochem 209: 298-305).

In vitro binding assays employ a mixture of components including a HUMAN CysLT2 GPCR polypeptide, which may be part of a fusion product with another peptide or polypeptide, e.g., a tag for detection or anchoring, and a sample suspected of containing a natural HUMAN CysLT2 GPCR binding target. A variety of other reagents such as salts, buffers, neutral proteins, e.g., albumin, detergents, protease inhibitors, nuclease inhibitors, and antimicrobial agents, may also be included. The mixture components can be added in any order that provides for the requisite bindings and incubations may be performed at any temperature which facilitates optimal binding. The mixture is incubated under conditions whereby the HUMAN CysLT2 GPCR specifically binds the suspected cellular binding target contained in the sample with a reference binding affinity. Incubation periods are chosen for optimal binding but are also minimized to facilitate rapid, high-throughput screening.

After incubation, the binding between the HUMAN CysLT2 GPCR and the suspected binding target is detected by any convenient way. For cell-free binding type assays, a separation step is often used to separate bound from unbound components. Separation may be effected by, for example, precipitation or immobilization, followed by washing by, e.g., membrane filtration or gel chromatography. For cell-free binding assays, one of the components usually comprises or is coupled to a label. The label may provide for direct detection such as, for example, radioactivity, luminescence, optical or electron density, or indirect detection such as an epitope tag or an enzyme. A variety of methods may be used to detect the label depending on the nature of the label and other assay components, e.g., through optical or electron density, radiative emissions, nonradiative energy transfers, or indirectly detected with antibody conjugates. A difference in the binding affinity of the HUMAN CysLT2 GPCR polypeptide to the suspected binding target as compared with the binding of the HUMAN CysLT2 GPCR polypeptide in the absence of the suspected binding target indicates that the test sample contains a suitable binding target for the HUMAN CysLT2 GPCR polypeptide. A difference, as used herein, is statistically significant and preferably represents at least a 50%, more preferably at least a 90% difference.

Alternatively, assays for binding targets for HUMAN CysLT2 GPCR can be performed using Biacore technology. Examples of how to use this technology can be found in U.S. Pat. No. 5,641,640 or are provided by the manufacturer of the instrument, Pharmacia, Piscataway, N.J.

The nucleic acids, cDNAs, oligonucleotides, polypeptides and antibodies for the HUMAN CysLT2 GPCR, which are the subject of this invention, provide a plurality of tools for studying GPCR-mediated activity and function in various cells and tissues and for diagnosing diseases and selecting activators, inhibitors or drugs with the potential to intervene in various disorders, diseases, or conditions in which altered HUMAN CysLT2 GPCR expression is implicated. The disorders, diseases, or conditions include, but are not limited to, asthma, inflammation, allergy, angiogenesis, respiratory distress syndrome, Crohn's disease, edema, high or low blood pressure growth, development, blood and bone homeostasis.

The present invention provides for HUMAN CysLT2 GPCR nucleic acid and their deduced amino acid sequences. These sequences were identified by their similarity to published or known open reading frames. Since ligands to GPCRs are associated with basic cellular processes such as cell proliferation, differentiation and cell signaling, these sequences are useful in the characterization of and delineation of normal and abnormal processes. The HUMAN CysLT2 GPCR nucleic acid sequences that are the subject of the present invention are useful in a variety of diagnostic assays used to evaluate the role of specific HUMAN CysLT2 GPCRs in normal, diseased, or therapeutically treated cells or tissues.

Purified HUMAN CysLT2 GPCR nucleic acid sequences have numerous applications in techniques known to those skilled in the art of molecular biology. These techniques include their use as hybridization probes, for chromosome and gene mapping, in PCR technologies, in the production of sense or antisense nucleic acids, in screening for new therapeutic molecules, and in screening for molecules capable of modulating HUMAN CysLT2 GPCR activity. These examples are not intended to be limiting. For example, antisense nucleic acid find usefulness in clinical settings wherein a receptor antagonist is called for but unavailable. The nucleic acid sequences disclosed herein may be used in molecular biology techniques that are currently under development or that have not yet been developed, provided that the new techniques rely on known properties of nucleic acid sequences, including but not limited to, such properties as the triplet genetic code and specific base pair interactions.

Due to the degeneracy of the genetic code, many HUMAN CysLT2 GPCR-encoding nucleic acid sequences may be produced. Some of these nucleic acid sequences will bear only minimal homology to the endogenous sequence of any known and naturally occurring HUMAN CysLT2 GPCR. However, Applicant specifically contemplates as his invention each and every possible variation of nucleic acid sequence that could be made by selecting combinations based on possible codon choices. These combinations are made in accordance with the standard triplet genetic code as applied to the nucleic acid sequence of naturally occurring HUMAN CysLT2 GPCR, and all such variations are to be considered as being specifically disclosed.

HUMAN CysLT2 GPCR nucleic acid sequences and their derivatives, variants or fragments thereof are preferably capable of identifying the nucleic acid sequence of the naturally occurring HUMAN CysLT2 GPCR. However, it may be desirable or advantageous to produce HUMAN CysLT2 GPCR-encoding nucleic acid sequences comprising a substantially different codon usage. By way of non-limiting example, codons can be selected to increase the level of expression of the HUMAN CysLT2 GPCR peptide in a particular expression host in accordance with the frequency with which particular codons are utilized by the host chosen. Another example in which a substantial alteration of the nucleic acid sequence encoding the HUMAN CysLT2 GPCR without altering the encoded amino acid sequence includes the production of RNA transcripts having more desirable properties, such as a longer half-life, than transcripts produced from the naturally occurring sequence.

Nucleic acid sequences encoding a HUMAN CysLT2 GPCR may be joined to a variety of other nucleic acid sequences by means of well established recombinant DNA techniques (see, for example, Sambrook J. et al., (1989) Molecular Cloning: A Laboratory Manual, Cold Spring Harbor Laboratory, Cold Spring Harbor, N.Y.; or Ausubel F. M. et al., (1989) Current Protocols in Molecular Biology, John Wiley & Sons, NY, NY). Useful sequences for joining to the HUMAN CysLT2 GPCR include, but are not limited to, DNA vectors such as plasmids, cosmids, lambda phage derivatives, phagemids, and BAC vectors. DNA vectors of interest include, but are not limited to, vectors for replication, expression, probe generation, sequencing, and genetic transfer. Vectors of interest may contain an origin of replication functional in at least one organism, convenient restriction enzyme sites, and selectable markers for one or more host cell systems. They may also contain DNA sequences that may be useful in molecular biology techniques which require homologous recombination events. Functional GPCRs may be expressed as fusion proteins (e.g. Wise, A., Carr, I. C., and Milligan, G. (1997) Biochem. J. 325, 17-21) or may even be split and expressed as partial proteins which then re-associate to generate a functional receptor (Ridge K D; Lee SS; Yao L L, Proc Natl Acad. Sci U S A Apr. 11,1995;92(8):3204-8).

Standard PCR such as described in U.S. Pat. Nos. 4,683,195; 4,800,195; and 4,965,188, provides additional uses for oligonucleotides based upon the HUMAN CysLT2 GPCR nucleic acid sequence. Such oligonucleotides are generally artificially synthesized, but they may be of recombinant origin or, in some applications, a mixture of both. Oligonucleotides generally are used in pairs and comprise two nucleic acid sequences, one with a sense orientation (5′ to 3′) and one with an antisense (3′ to 5′). They are generally used under optimized conditions for the purpose of identifying a specific gene or for diagnostic use. In addition, the same two oligonucleotide pairs, a pair of “nested” oligonucleotides, or a pool of degenerate oligonucleotides may be used under less stringent or optimized conditions for identification and/or quantitation of closely related DNA or RNA sequences.

Other useful PCR-based techniques include (1) Inverse PCR, which is the first method to report successful acquisition of unknown sequences starting with primers based on a known region (Triglia, T. et al (1988) Nucleic Acids Res 16:8186); (2) Capture PCR (Lagerstrom M. et al (1991) PCR Methods Applic 1:111-19) which is a method for PCR amplification of DNA fragments adjacent to a known sequence in human and YAC DNA; (3) targeted gene walking (Parker J. D. et al (1991; Nucleic Acids Res 19:3055-60) which is a method for targeted gene walking which permits retrieval of unknown sequence; and (4) Capillary Electrophoresis which is a new method for analyzing either the size or the nucleic acid sequence of PCR products.

Another aspect of the subject invention is to provide for HUMAN CysLT2 GPCR hybridization probes which are capable of hybridizing with naturally occurring nucleic acid sequences encoding HUMAN CysLT2 GPCR. The stringency of the hybridization conditions will determine whether the probe identifies only the native nucleic acid sequence of that specific HUMAN CysLT2 GPCR or sequences of closely related molecules. Demonstrating specific hybridization generally requires stringent conditions, for example, hybridizing in a buffer comprising 30% formamide in 5× SSPE (0.18 M NaCl, 0.01 M NaPO₄, pH 7.7, 0.001 M EDTA) buffer at a temperature of 42° C. and remaining bound when subject to washing at 42° C. with 0.2× SSPE; preferably hybridizing in a buffer comprising 50% formamide in 5× SSPE buffer at a temperature of 42° C. and remaining bound when subject to washing at 42° C. with 0.2× SSPE buffer at 42° C. HUMAN CysLT2 GPCR homologs can also be distinguished from one another using alignment algorithms, such as BLASTX (Altschul, et al., (1990) Basic Local Alignment Search Tool, J. Mol. Biol. 215:403-410). If degenerate HUMAN CysLT2 GPCR nucleic acid sequences of the subject invention are used for the detection of related HUMAN CysLT2 GPCR encoding sequences, they should preferably contain at least 50% of the nucleotides of the sequences presented herein. Hybridization probes of the subject invention may be derived from the nucleic acid sequence of HUMAN CysLT2 GPCR, or from surrounding or included genomic sequences comprising untranslated regions such as promoters, enhancers and introns. Such hybridization probes may be labeled with appropriate reporter molecules. Means for producing specific hybridization probes for HUMAN CysLT2 GPCR include oligonucleotide labeling, nick translation, end-labeling or PCR amplification using a labeled oligonucleotide. Alternatively, the cDNA sequence may be cloned into an appropriate vector for the production of an mRNA probe. Such vectors are known to those skilled in the art and are commercially available. They may be used to synthesize RNA probes in vitro by the addition of an appropriate RNA polymerase such as T7, T3 or SP6 and appropriately labeled nucleotides. Several companies, including Pharmacia Biotech, Piscataway, N.J.; Promega, Madison, Wis.; and US Biochemical Corp., Cleveland, Ohio; supply commercial kits and protocols for these procedures.

It is also possible to produce a DNA sequence, or a portion or fragment thereof, entirely by synthetic chemistry using laboratory equipment familiar to the skilled artisans. The source of information for producing the synthetic sequence may be derived from the known homologous sequence from closely related organisms. After synthesis, the nucleic acid sequence can be used alone or joined with another known sequence and inserted into one of the many available DNA vectors and their respective host cells using techniques well known in the art. Moreover, synthetic chemistry may be used to introduce specific mutations into the nucleic acid sequence. Alternatively, a portion of sequence in which a mutation is desired can be synthesized and recombined with a portion of an existing genomic or recombinant sequence.

The HUMAN CysLT2 GPCR nucleic acid sequences can be used individually, in panels or arrays, or in diagnostic tests or assays to detect disorders or disease processes that are associated with abnormal levels of HUMAN CysLT2 GPCR expression. By way of non-limiting example, the nucleic acid sequence can be added to a sample to be tested (e.g. a body fluid such as blood, plasma, synovial fluid, or CSF or a cell or tissue, including homogenates of cells or tissues), obtained from a patient, under hybridizing conditions. After an incubation period, the sample is washed with a compatible fluid which may or may not contain a reporter molecule which will bind the specific nucleic acid. After the compatible fluid is rinsed off, the reporter molecule is quantitated and compared with a standard for that fluid, cell or tissue. If HUMAN CysLT2 GPCR expression is significantly different from the standard, the assay indicates the presence of a disorder or disease. The form of such methods may include Northern analysis, dot blot or other membrane based technologies, dip stick, pin or chip technologies, PCR, ELISAs or other multiple sample format technologies.

A same or similar assay format is applicable in evaluating the efficacy of a particular therapeutic treatment regime. For example, it may be used in evaluating efficacy in animal studies, in human clinical trials, or in monitoring the treatment of an individual patient. In this application, standard expression must be established for use as a basis of comparison with the test samples. Samples from the experimental animals or patients that are affected by the disorder or disease are combined with the nucleic acid sequence to evaluate the difference from the standard or normal expression profile. Next, a therapeutic agent is administered to the experimental animal or patient and a treatment profile is obtained. The assay is evaluated to determine whether or not the profile progresses toward or returns to the standard pattern. Successive treatment profiles may be used to show the efficacy of treatment over a period of time.

The nucleic acid sequence for HUMAN CysLT2 GPCR can also be used to generate probes for genomic mapping of the native sequence to a particular chromosome or to a specific region of a chromosome using techniques well known to the skilled artisan. These techniques include, but are not limited to, in situ hybridization to chromosomal spreads (Verma et al (1988) Human Chromosomes: A Manual of Basic Techniques, Pergamon Press, NY, N.Y.), flow-sorted chromosomal preparations, or artificial chromosome constructions such as yeast artificial chromosomes (YACs), bacterial artificial chromosomes (BACs), bacterial P1 constructions or single chromosome cDNA libraries.

In situ hybridization of chromosomal preparations and physical mapping techniques such as linkage analysis using established chromosomal markers are invaluable in extending genetic maps. Examples of genetic maps can be found in the 1994 Genome Issue of Science (265:1981f). Genetic mapping provides invaluable information to investigators searching for disease-associated genes using gene discovery techniques. Once a disease or syndrome has been generally localized by genetic linkage to a particular genomic region, any sequences mapping to that area represent genes that may be suitable for further investigation. In addition, the nucleic acid sequences of the subject invention may also be used to detect differences in the chromosomal location of nucleic acid sequences due to, for example, translocation or inversion between normal and carrier or affected individuals.

The partial nucleic acid sequence encoding a particular HUMAN CysLT2 GPCR may be used to produce an amino acid sequence using well known methods of recombinant DNA technology. The amino acid or polypeptide or polypeptide comprising a fragment thereof may be expressed in a variety of host cells, either prokaryotic or eukaryotic. Host cells may be from the same species from which the nucleic acid sequence was derived or from a different species.

Cells transformed with a HUMAN CysLT2 GPCR nucleic acid sequence may be cultured under conditions suitable for the expression and recovery of a polypeptide from cell culture. The receptor may be isolated in a detergent solubilized form and reassembled into membranes or it may be isolated as membrane fragments or vesicles following physical disruption of transfected cells. Other recombinant constructions may join HUMAN CysLT2 GPCR nucleic acid to a nucleic acid sequence encoding a polypeptide domain which will facilitate protein purification, for example, the Fc region of an antibody or a tag sequence such as a HIS tag.

Antibodies specific for HUMAN CysLT2 GPCR may be produced by inoculation of an appropriate animal with an antigenic fragment of the HUMAN CysLT2 GPCR polypeptide. Although an amino acid sequence or oligopeptide or polypeptide used for antibody induction does not require biological activity, it must be immunogenic. HUMAN CysLT2 GPCR polypeptides or fragments thereof used to induce specific antibodies may have a polypeptide sequence consisting of at least five amino acids and preferably at least 10 amino acids. Short stretches of amino acid sequence may be fused with those of another protein or polypeptide such as keyhole limpet hemocyanin, and the chimeric polypeptide used for antibody production. Alternatively, the polypeptide may be of sufficient length to contain an entire domain of HUMAN CysLT2 GPCR. An antibody is specific for HUMAN CysLT2 GPCR if it is produced against an epitope of the polypeptide and binds to at least part of the natural or recombinant protein. Antibody production includes not only the stimulation of an immune response by injection into animals, but also analogous processes such as the production of synthetic antibodies, the screening of recombinant immunoglobulin libraries for specific-binding molecules (Orlandi R. et al (1989) PNAS 86:3833-3837, or Huse W. D. et al (1989) Science 256:1275-1281), or the in vitro stimulation of lymphocyte populations. Current technology (Winter G. and Milstein C. (1991) Nature 349:293-299) provides for a number of highly specific binding reagents based on the principles of antibody formation. These techniques may be adapted to produce molecules which specifically bind HUMAN CysLT2 GPCR. Antibodies or other appropriate molecules generated against a specific immunogenic peptide fragment or oligopeptide can be used in Western analysis, enzyme-linked immunosorbent assays (ELISA) or similar tests to establish the presence of or to quantitate amounts of HUMAN CysLT2 GPCR active in normal, diseased, or therapeutically treated cells or tissues. Wholly human monoclonal antibodies are also provided for and can be made as described in U.S. Pat. No. 5,939,598, issued Aug. 17, 1999 and assigned to Abgenix, Inc.

The examples below are provided to illustrate the subject invention. These examples are provided by way of illustration and are not included for the purpose of limiting the invention.

### EXAMPLES

### Example 1

### Identification of HUMAN CysLT2 GPCR

An extensive database (>4000 sequences) of all known GPCR protein sequences was compiled. The database was expanded by several rounds of homology search, BLASTp BLAST 2.0 was obtained from the NCBI ftp site (ftp://ncbi.nim.nih.gov/blast/executables). This homology search was performed against public protein sequences from GenBank. The positions of putative transmembrane segments was annotated for each family member using a combination of homology (matching transmembrane positions to those of the closest homologue), hydrophobicity and alignment of key conserved residues to general models (e.g. Baldwin J M; Schertler G F; Unger V M, J Mol Biol Sep. 12,1997;272(1):144-64). In addition to BLAST search, the CLUSTALW algorithm (CLUSTALW 1.7, Nucleic Acids Research, 22(22):4673-4680), which was downloaded from www.csc.fi/molbio/progs/clustalw/clustalw.html), was also used in some cases to align sequences for annotation of transmembrane regions.

New GPCR homologues were identified from human genomic DNA sequence as follows: Both finished and unfinished high throughput human genomic DNA sequence was downloaded weekly from the NCBI database which can be accessed via the internet at the following URL: ftp://ncbi.nlm.nih.gov/genbank/genomes/H_sapiens. The DNA sequences were converted into predicted proteins using the GenScan program (GenScan, Burge, C. & Karlin, S., 1997, Prediction of complete gene structures in human genomic DNA. J. Mol. Biol. 268, 78-94; Burge, C. B. and Karlin, S., 1998, Finding the genes in genomic DNA. Curr. Opin. Struct. Biol. 8, 346-354, licensed from Stanford University) set for human/vertebrate/Drosophila bias. All proteins greater than 20 amino acids in length that were predicted using either optimal or suboptimal exons (see annotation of GenScan for a description of optimal and suboptimal exon prediction) (cutoff=0.1) were included Each of the predicted proteins were compared using BLASTp to the GPCR database created as described supra using only those regions of each sequence in the GPCR database that extend from the first through the seventh transmembrane domain inclusive (Tms 1-7). Each predicted protein which showed homology to any member of the database (cutoff=e<10-4, e=expected value as defined by the BLAST program) was examined. HUMAN CysLT2 GPCR was initially identified as an open reading frame from a BAC clone (RP11-108P5 GenBank acc. #AL137118 mapping on chromosome 13 between q14.12 and q21.1).

The identified intronless open reading frame was PCR-amplified from genomic DNA using the following oligonucleotides:

CysLT1-like-atg1F:
CCACCATGGAGAGAAAATTTATGTCCTTGCAACCATC
CysLT1L-ter2:
AGCTCCTTATACTCTTGTTTCCTTTCTCAACCACACACTAAC

These same primers also produced a PCR product from cDNA derived from human placental RNA demonstrating that this open reading frame is present on mRNA. The resulting PCR fragment was cloned into a series of expression vectors for functional analyses as described infra.

### Example 2

### Comparison of CysLT1 and HUMAN CysLT2 GPCR Amino Acid Sequences.

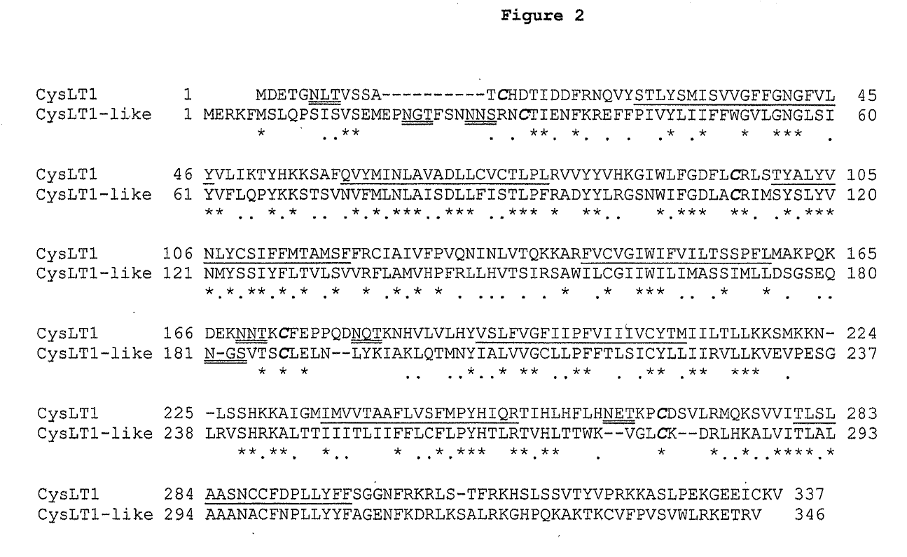

A comparison of the CysLT1 (gi5729798; Lynch K R, et al., Nature 399 :789; Sarau H M, et al., Mol Pharmacol. 1999, 56:657) and HUMAN CysLT2 GPCR amino acid sequences was performed using the ClustalW algorithm imbedded in MacVector 6.5.1 (Oxford Molecular Group, plc). The results of this comparison are shown in FIG. 2. The high degree of homology, especially within the transmembrane domains, indicates that HUMAN CysLT2 GPCR identified supra is likely to be a GPCR.

### Example 3

### Expression Pattern of HUMAN CysLT2 GPCR

Expression levels of HUMAN CysLT2 GPCR mRNAs in human tissues were ascertained by TaqMan analysis. The abundance of mRNA was determined using the quantitative RT-PCR “TaqMan” procedure (Lie, Y. S. and Petropoulos, C. J., “Advances in quantitative PCR technology: 5′ nuclease assays”, Curr Opin Biotechnol 9 (1998): 43-48.) with a PE ABI PRISM 7700 Sequence Detection System instrument (PE Biosystems, Foster City, Calif.).

This method employs two oligonucleotides spaced relatively close to each other to PCR amplify a portion of the message from cDNA and a third “probe” oligonucleotide co-labeled with a fluorophore and quencher at each end. When the level of the PCR product builds up to a sufficient level, a significant fraction of the fluorophore is released by a “nick-translation” exonucleolytic activity of the polymerase. The released fluorophore becomes highly fluorescent by being dissociated from the quenching moiety. The abundance of a specific mRNA is determined by reading fluorescence during the course of the PCR reaction: samples containing more abundant messages taking fewer PCR cycles to release probe fluorescence, while samples containing the same message in lower abundance will require more cycles.

For TaqMan analysis of HUMAN CysLT2 GPCR the following oligonucleotides were employed:

1. CysLT1L-Taq1F: CCGTGCTGAGTGTTGTGCG
2. CysLT1L-Taq1R: CCTGATGCTGGTGACATGCAG
3. CysLT1L-TaqMan1: CCTGGCAATGGTTCACCCCTTTCG

The TaqMan PCR reactions were run on a Perkin Elmer ABI PRISM 7700 Sequence Detection System instrument. MicroAmp (Perkin Elmer) optical 96-well plates and optical caps were used. Each reaction had a final volume of 25 μl and the following concentrations of components: 1× TaqMan buffer A, 4 mM MgCl₂, 200 μM of each of dATP, dCTP, dGTP, and 400 μM dUTP, 300 nM of each of forward (CysLT1L-Taq1F) and reverse (CysLT1L-Taq1R) primers, 200 nM of TaqMan probe, 5% DMSO, 10% glycerol, 0.025 U/μl AmpliTaq Gold, and 1 U/μl AmpErase UNG. The PCR cycling conditions were as follows: 2 min. at 50° C., 10 min. at 95° C., followed by 40 two-step cycles of 15 sec at 95° C. and 1 min. at 60° C. The TaqMan probe had a 6-FAM 5′-Fluorescent label and TAMRA 3′-label that acts as a quencher.

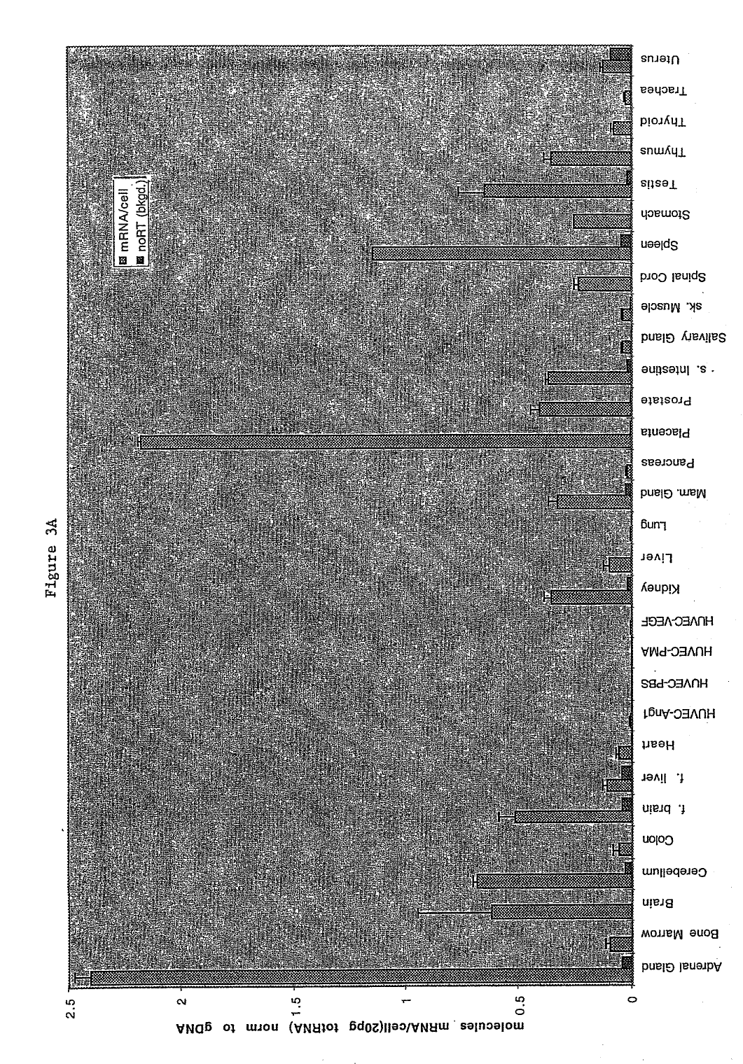

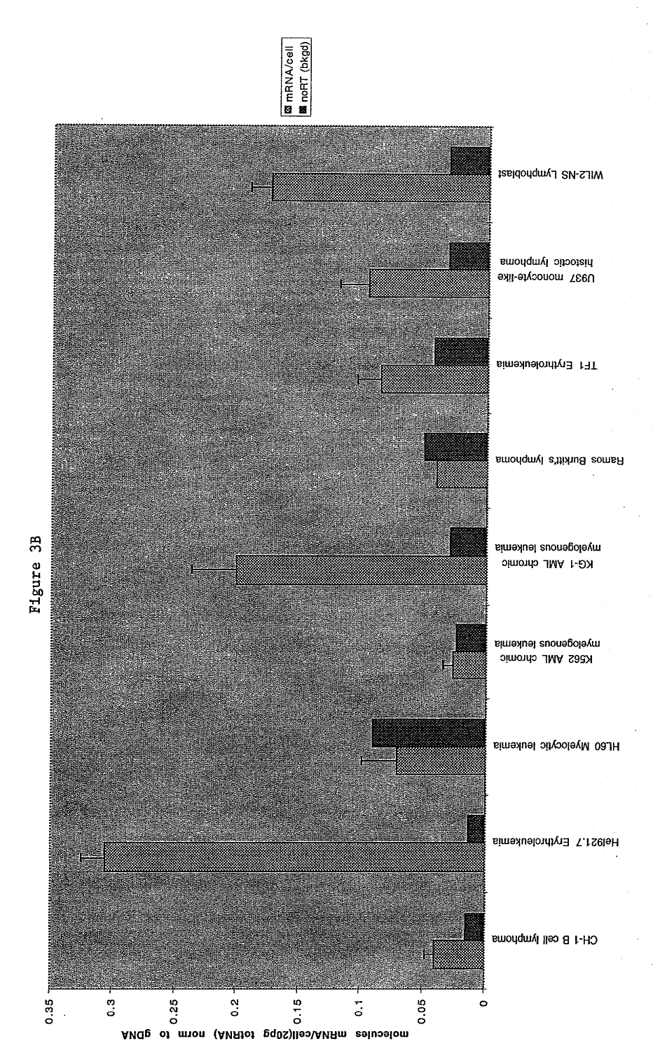

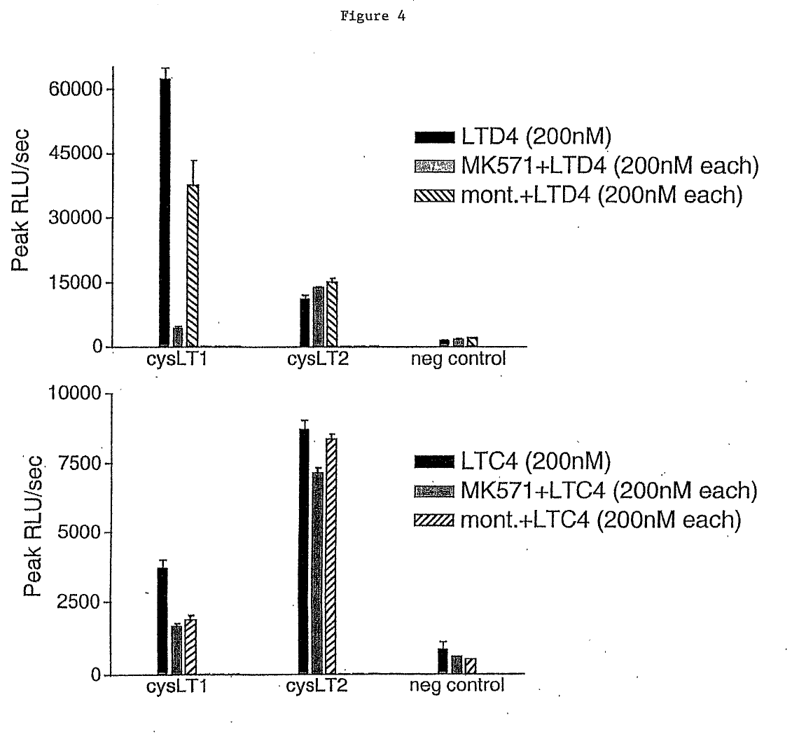

The results of this analysis are set forth in FIGS. 3A-3B.

### Example 4

### Activation of HUMAN CysLT2 GPCR by Cysteinyl Leukotrienes.

In order to evaluate receptor activation, a bioluminescence assay was used to detect intracellular calcium release in response to cysteinyl leukotrienes (LTC4 and LTD4, Biomol). In this assay, light is produced by a chemical reaction of a specific photoprotein within the cells and does not require illumination, and thus, it is sensitive and relatively free of background. The photoprotein aequorin, originally isolated from luminescent jellyfish, has proven to be a highly sensitive and quantitative bioluminescence Ca²⁺ indicator in cells.

The aequorin complex contains a 22,000 MW apoaequorin protein, oxygen and the luminophore, coelenterazine. When three Ca²⁺ ions bind to this complex, coelenterazine is oxidized to coelenteramide, releasing carbon dioxide and light (emission maximum ˜466 nm). Aequorin has a broad detection range, allowing the measurement of Ca²⁺ concentrations from ˜0.1 μM to >100 μM.

The HUMAN CysLT2 GPCR and apoaequorin cDNAs were transiently co-expressed in cells (the aequorin is targeted for expression in mitochondria). Once cells have been transfected with these expression vectors, they are incubated in a medium containing cell-permeant coelenterazine which reconstitutes the aequorin complex. After formation of the active aequorin complex, intracellular Ca²⁺ release caused by activation of the HUMAN CysLT2 GPCR is measured by assaying cells for light production using a luminometer.

Assay Method

COS-7 cells were trypsinized and plated at 1.5×10⁵ cells/well in 6 well tissue culture plates. The cells were grown overnight at 37° C. in DMEM supplemented with 10% FBS, 1% glutamine and 1% non essential amino acids. The next day, the cells were transfected with HUMAN CysLT2 GPCR sequence that had been previously subcloned into the expression vector pcDNA 3.1 and aequorin (mitochondrial targeted) that had also been subcloned into the expression vector pcDNA3.1. For comparison purposes the human CysLT1 recptor (Sarau H M, Ames R S, Chambers J, Ellis C, Elshourbagy N, Foley J J, Schmidt D B, Muccitelli R M, Jenkins O, Murdock P R, Herrity N C, Halsey W, Sathe G, Muir A I, Nuthulaganti P, Dytko G M, Buckley P T, Wilson S, Bergsma D J, Hay D W, Mol Pharmacol. 1999 September;56(3):657-63; Lynch K R, O'Neill G P, Liu Q, Im D S, Sawyer N, Metters K M, Coulombe N, Abramovitz M, Figueroa D J, Zeng Z, Connolly B M, Bai C, Austin C P, Chateauneuf A, Stocco R, Greig G M, Kargman S, Hooks S B, Hosfield E, Williams D L Jr, Ford-Hutchinson A W, Caskey C T, Evans J F, Nature. Jun. 24, 1999;399(6738):789-93.; GenBank accession number: NP_{—}006630) also subcloned into pcDNA3.1, or pcDNA3.1 containing a “dummy” insert were transfected with aequorin into parallel cell cultures. The transfection was done using Lipofectamine Plus (Life Technologies) as follows:

DNA (0.75mg each) is added to 100 ml medium (without serum); 6 ml of Lipofectamine Plus reagent is added, mixed, and incubated for 15 min.

In a second tube, 4 ml Lipofectamine reagent is diluted into 100 ml medium without serum and mixed.

The pre-complexed DNA and diluted Lipofectamine reagent are combined, mixed, and incubated for 15 min.

The medium from each well of the 6 well tissue culture plate containing the COS-7 cells is removed and replaced with 0.8 ml complete medium (supplemented with 5% FBS). The transfection complex is added, mixed gently and incubated at 37° C., 5% CO₂ for 3.5 hrs.

To recover the transfected cells and perform the Aequorin assay, the following steps were performed:

The transfection medium was removed and complete medium supplemented with 1% penicillin/streptomycin was added and the cells were incubated at 37° C., 5% CO₂ for 48 hrs.

The medium from cells was removed and Ham's F-12 medium supplemented with 25 mM HEPES, 0.1% FBS with 8 μM coelentrazine cp (Molecular probes) was added and the cells were incubated at 37° C., 5% CO₂ for 2 hrs.

The cells were scraped from the wells and washed once with Ham's F-12 supplemented with 25 mM HEPES and 0.1% FBS.

The cells were resuspended at 2-4×10⁵ cells/ml. 100 μl of cells were added to each well of a solid white 96 well plate (EG&G Wallac) and incubated at room temperature for 30 min.

A luminometer (TR717, Tropix, PE Applied Biosystems) was used to detect the light emitted from the assay. The TR717 is a microplate luminometer that gives a digital signal by directly identifying individual emitted photons via a photomultiplier tube (PMT). The instrument has an injector located directly over the well being measured to allow for flash detection. To obtain accurate flash measurements, an injection and measurement can be initiated concurrently with this injector. There is also a second injector located over the well next in line to be measured. This allows for pre-injection into one well while the well prior to it is being measured by the PMT.

The test compound (LTC4 or LTD4, final concentration of 200 nM) in PBS was pre-injected (25 μl ) into the wells (one well at a time) and after a delay of 1.6 seconds the emitted light was measured for 20 seconds. A second injection (25 μl ) of 0.9% Triton X100 in PBS was used to lyse the cells and obtain a total count of emitted light. For testing the antagonist effects of MK571 or Singulair, wells were injected with the antagonist (final concentration of 200 nM, diluted out of a 400 to 500×stock solution in ethanol) 10 seconds prior to agonist injection.

Results

COS-7 cells expressing HUMAN CysLT2 GPCR, CysLT1 or no leukotriene receptor and aequorin were assayed for aequorin activation in response to 200 nM of LTC4 or LTD4, either alone or in the presence of 200 nM of the CysLT1 specific antagonists MK571 or Singulair (montelukast). While both CysLT receptors responded to both LTC4 and LTD4, the rank order of their effects was different. CysLT1 was more strongly activated by LTD4 and CysLT2 was more strongly activated by LTC4. In addition the action of both LTC4 and LTD4 on the CysLT1 receptor was effectively blocked by MK571 and Singulair (montelukast) whereas neither antagonist significantly inhibited the effects of LTC4 or LTD4 on HUMAN CysLT2 GPCR.

What is claimed is:

1. Isolated HUMAN CysLT2 GPCR polypeptide.

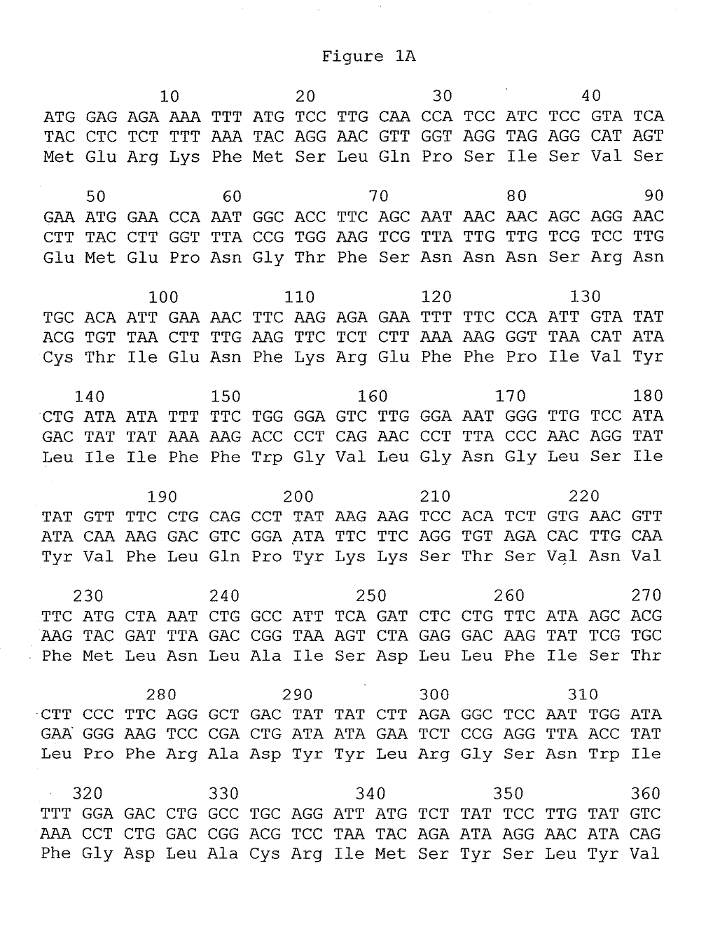

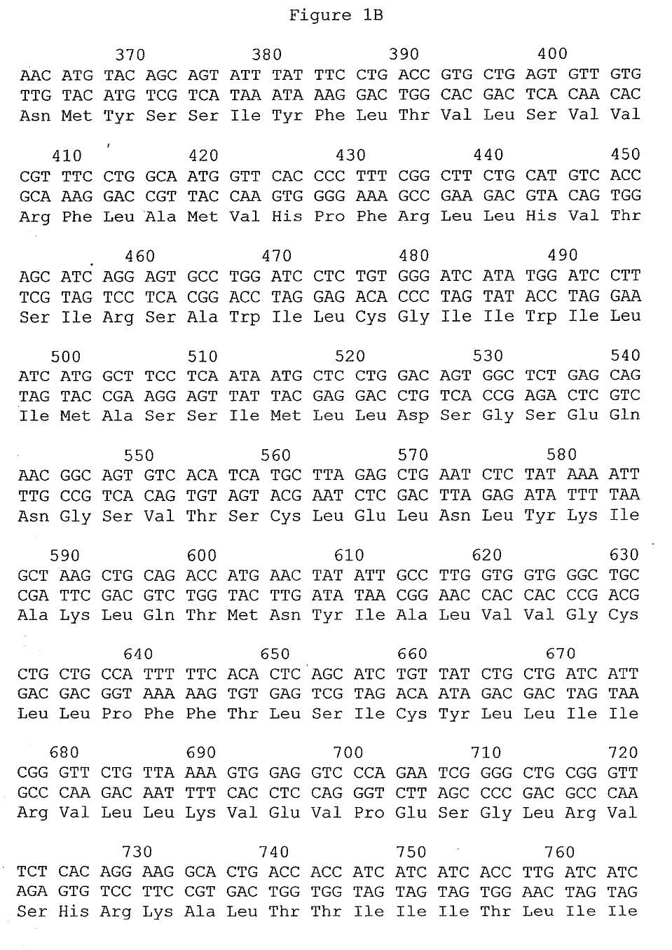

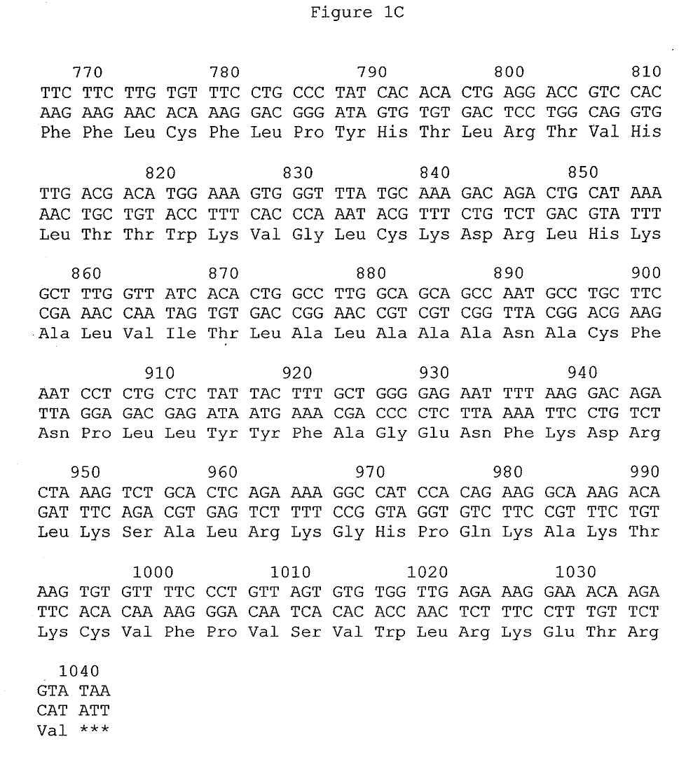

2. Isolated HUMAN CysLT2 GPCR polypeptide comprising the amino acid sequence as set forth in FIGS. 1A-1C.

3. Isolated HUMAN CysLT2 GPCR polypeptide comprising a polypeptide fragment or derivative thereof.

4. The isolated HUMAN CysLT2 GPCR polypeptide of claim 1 or 2 encoded by the nucleic acid molecule as set forth in FIGS. 1A-1C.

5. An isolated nucleic acid molecule having a sequence selected from the group consisting of:
(a) the nucleotide sequence comprising the coding region of the HUMAN CysLT2 GPCR as set forth in FIGS. 1A-1C; or
(b) a nucleotide sequence that hybridizes under stringent conditions to the complement of the nucleotide sequence of (a) and which encodes HUMAN CysLT2 GPCR, wherein said stringent conditions are 30% formamide in 5× SSPE (0.18 M NaCl, 0.01 M NaPO₄, pH 7.7, 0.001 M EDTA) buffer at a temperature of 42° C. and remaining bound when subject to washing at 42° C. with 0.2× SSPE; or
(c) a nucleotide sequence which, as a result of the degeneracy 25 of the genetic code, differs from the nucleic acid of (a) or (b) and which encodes HUMAN CysLT2 GPCR.

6. A vector which comprises a nucleic acid of claim 5.

7. A vector according to claim 6, wherein the nucleic acid molecule is operatively linked to an expression control sequence capable of directing its expression in a host cell.

8. A vector according to claim 6 or 7, which is a plasmid.

9. A host-vector system for the production of HUMAN CysLT2 GPCR which comprises a vector of claim 6 or 7, in a host cell.

10. A host-vector system according to claim 9, wherein the host cell is a bacterial, yeast, insect, amphibian or mammalian cell.

11. Isolated nucleic acid molecules which are complementary to the HUMAN CysLT2 GPCR nucleotide sequences of claim 5.

12. A chimeric protein which comprises extracellular portions of the CysLT2 GPCR protein fused to an immunoglobulin, an immunoglobulin constant region or a fragment thereof.

13. The chimeric protein of claim 12 wherein such extracellular portions comprise an extracellular domain of the amino terminus of CysLT2, an extracellular domain located between the second and third transmembrane domains, an extracellular domain located between the fourth and fifth transmembrane domains, an extracellular domain located between the fifth and sixth transmembrane domains, or any combination thereof.

14. The chimeric protein of claim 13 wherein such extracellular domain of the amino terminus comprises amino acid residues 1-41 of FIGS. 1A-1C.

15. The chimeric protein of claim 13 wherein such an extracellular domain between the second and third transmembrane domains comprises amino acid residues 94-115 of FIGS. 1A-1C.

16. The chimeric protein of claim 13 wherein such an extracellular domain between the fourth and fifth transmembrane domains comprises amino acid residues 175-203 of FIGS. 1A-1C.

17. The chimeric protein of claim 13 wherein such an extracellular domain between the sixth and seventh transmembrane domains comprises amino acid residues 268-289 of FIGS. 1A-1C.

18. A method of producing HUMAN CysLT2 GPCR which comprises growing cells of a host-vector system of claim 10, under conditions permitting production of the HUMAN CysLT2 GPCR, and recovering the HUMAN CysLT2 GPCR so produced.

19. A polypeptide produced by the method of claim 18.

20. An antibody which specifically binds the HUMAN CysLT2 GPCR of claim 1, 2 or 3.

21. An antibody according to claim 20, which is a monoclonal antibody.

22. An antibody according to claim 21, which is a wholly human monoclonal antibody.

23. A composition comprising HUMAN CysLT2 GPCR according to 1, 2 or 3, and a carrier.

24. A composition comprising an antibody according to claim 20, and a carrier.

25. HUMAN CysLT2 GPCR according to claim 1, 2 or 3 for use in a method of treatment of the human or animal body, or in a method of diagnosis.

26. An antibody according to claim 20 for use in a method of treatment of the human or animal body, or in a method of diagnosis.

27. A composition according to claim 23 for use in a method of treatment of the human or animal body, or in a method of diagnosis.

28. A composition according to claim 24 for use in a method of treatment of the human or animal body, or in a method of diagnosis.

29. A method of identifying a HUMAN CysLT2 GPCR binding partner comprising:
(a) contacting HUMAN CysLT2 GPCR polypeptide with a test sample suspected of containing a HUMAN CysLT2 GPCR binding partner;
(b) contacting HUMAN CysLT2 GPCR polypeptide with a control sample that does not contain a HUMAN CysLT2 GPCR binding partner;
(c ) comparing the amount of binding in (a) to the amount of binding in (b) wherein a greater amount of binding in (a) is indicative of the presence of a HUMAN CysLT2 GPCR binding partner in the test sample.

30. A binding assay for identifying an agent which specifically binds to a HUMAN CysLT2 GPCR comprising contacting cells expressing on their cell surface a HUMAN CysLT2 GPCR with the agent under conditions suitable for binding, and detecting specific binding of the agent to the HUMAN CysLT2 GPCR.

31. A competitive binding assay useful for identifying an agent which specifically binds to HUMAN CysLT2 GPCR comprising:
a) obtaining cells expressing on their cell surface HUMAN CysLT2 GPCR;
b) contacting the cells with a first agent known to bind to HUMAN CysLT2 GPCR;
c) detecting the amount of binding of the first agent in (b) to HUMAN CysLT2 GPCR;
d) contacting (b) with a second agent whose ability to specifically bind to HUMAN CysLT2 GPCR is unknown;
e) detecting the amount of binding of the first agent in (d) to HUMAN CysLT2 GPCR;
f) comparing the amount of binding of the first agent detected in (c) with the amount of binding of the first agent detected in (e) wherein a decrease in the amount of binding of the first agent is indicative of the second agent's ability to specifically bind to HUMAN CysLT2 GPCR.

32. A competitive binding assay useful for identifying an agent which specifically binds to HUMAN CysLT2 GPCR comprising:
a) preparing a sample comprising HUMAN CysLT2 GPCR;
b) contacting the sample with a first agent known to bind to HUMAN CysLT2 GPCR;
c) detecting the amount of binding of the first agent in (b) to HUMAN CysLT2 GPCR;
d) contacting (b) with a second agent whose ability to specifically bind to HUMAN CysLT2 GPCR is unknown;
e) detecting the amount of binding of the first agent in (d) to HUMAN CysLT2 GPCR;
f) comparing the amount of binding of the first agent detected in (c) with the amount of binding of the first agent detected in (e) wherein a decrease in the amount of binding of the first agent is indicative of the second agent's ability to specifically bind to HUMAN CysLT2 GPCR.

33. A competitive binding assay useful for identifying an agent which specifically binds to HUMAN CysLT2 GPCR comprising:
a) obtaining cells expressing on their cell surface HUMAN CysLT2 GPCR;
b) contacting a test sample of the cells of (a) with a first agent whose ability to specifically bind to HUMAN CysLT2 GPCR is unknown;
c) contacting the test sample of the cells of (b) with a second agent known to bind to HUMAN CysLT2 GPCR;
d) contacting a control sample of the cells of (a) with the second agent known to bind to HUMAN CysLT2 GPCR;
e) detecting the amount of binding of the second agent in (c) to HUMAN CysLT2 GPCR;
f) detecting the amount of binding of the second agent in (d) to HUMAN CysLT2 GPCR;
g) comparing the amount of binding of the second agent detected in (e) with the amount of binding of the second agent detected in (f) wherein a lesser amount of binding of the second agent in (e) is indicative of the first agent's ability to specifically bind to HUMAN CysLT2 GPCR.

34. A competitive binding assay useful for identifying an agent which specifically binds to HUMAN CysLT2 GPCR comprising:
a) preparing a sample comprising HUMAN CysLT2 GPCR;
b) contacting a test sample of the sample of (a) with a first agent whose ability to specifically bind to HUMAN CysLT2 GPCR is unknown;
c) contacting the test sample of (b) with a second agent known to bind to HUMAN CysLT2 GPCR;
d) contacting a control sample of the sample of (a) with the second agent known to bind to HUMAN CysLT2 GPCR;
e) detecting the amount of binding of the second agent in (c) to HUMAN CysLT2 GPCR;
f) detecting the amount of binding of the second agent in (d) to HUMAN CysLT2 GPCR;
g) comparing the amount of binding of the second agent detected in (e) with the amount of binding of the second agent detected in (f) wherein a lesser amount of binding of the second agent in (e) is indicative of the first agent's ability to specifically bind to HUMAN CysLT2 GPCR.

35. A competitive binding assay useful for identifying an agent which specifically binds to HUMAN CysLT2 GPCR comprising:
a) obtaining cells expressing on their cell surface HUMAN CysLT2 GPCR;
b) contacting a test sample of the cells of (a) with a first agent known to bind to HUMAN CysLT2 GPCR and with a second agent whose ability to bind to HUMAN CysLT2 GPCR is unknown;
c) contacting a control sample of the cells of (a) with the first agent known to bind to HUMAN CysLT2 GPCR;
d) detecting the amount of binding of the first agent in (b) to HUMAN CysLT2 GPCR;
e) detecting the amount of binding of the first agent in (c) to HUMAN CysLT2 GPCR;
f) comparing the amount of binding of the first agent detected in (d) with the amount of binding of the first agent detected in (e) wherein a decrease in the amount of binding of the first agent in (d) is indicative of the second agent's ability to specifically bind to HUMAN CysLT2 GPCR.

36. A competitive binding assay useful for identifying an agent which specifically binds to HUMAN CysLT2 GPCR comprising:
a) preparing a sample comprising HUMAN CysLT2 GPCR;
b) contacting a test sample of the sample of (a) with a first agent known to bind to HUMAN CysLT2 GPCR and with a second agent whose ability to bind to HUMAN CysLT2 GPCR is unknown;
c) contacting a control sample of the sample of (a) with the first agent known to bind to HUMAN CysLT2 GPCR;
d) detecting the amount of binding of the first agent in (b) to HUMAN CysLT2 GPCR;
e) detecting the amount of binding of the first agent in (c) to HUMAN CysLT2 GPCR;
f) comparing the amount of binding of the first agent detected in (d) with the amount of binding of the first agent detected in (e) wherein a decrease in the amount of binding of the first agent in (d) is indicative of the second agent's ability to specifically bind to HUMAN CysLT2 GPCR.

37. The assay of claim 30, 31, 32, 33, 34, 35, or 36 wherein the detection of the binding of the agent to HUMAN CysLT2 GPCR is accomplished by any one of the methods selected from the group consisting of radioactive detection, fluorescence detection, chromogenic detection, mass spectroscopy, and plasmon resonance.

38. The assay of claims 31, 32, 35, or 36 wherein the first agent is LTC4 or LTD4 or a derivative thereof.

39. The assay of claims 33 or 34 wherein the second agent is LTC4 or LTD4 or a derivative thereof.

40. The assay of claim 31, 32 or 35 wherein the detection of specific binding of the agent to HUMAN CysLT2 GPCR is accomplished by detecting a biological response.

41. The assay of claim 31, 32, or 35 wherein the biological response is selected from the group consisting measuring Ca²⁺ ion flux, cAMP, IP₃, PIP₃and transcription of reporter genes.

42. The assay of claim 30, 31, 33 or 35 wherein the cell expressing HUMAN CysLT2 GPCR is a mammalian cell.

43. The assay of claim 42 wherein the mammalian cell is a COS-7 cell, a 293 human embryonic kidney cell, a NIH 3T3 cell, or Chinese hamster ovary (CHO) cell.
